(19) United States
(12) Patent Application Publication
(10) Pub. No.: US 2022/0031935 A1
(43) Pub. Date: Feb. 3, 2022

(54) SYRINGE HOLDING DEVICE AND ORGANIZER

(71) Applicant: Wendy Bowers, Camarillo, CA (US)

(72) Inventor: Wendy Bowers, Camarillo, CA (US)

(21) Appl. No.: 17/387,820

(22) Filed: Jul. 28, 2021

Related U.S. Application Data
(60) Provisional application No. 63/057,428, filed on Jul. 28, 2020.

(51) Int. Cl. A61M 5/00

(52) U.S. Cl. CPC A61M 5/008; A61M 2209/08

(57) ABSTRACT

Devices for holding syringes are disclosed that can hold the syringes in a manner to allow for safe and easy access and use of the syringes by a practitioner. The holders according to the present invention can comprise a holder body with a plurality of cradles or openings for holding a syringe body, with the cradles/openings arranged to allow for fast and convenient mounting and removal of syringes to and from the holder. On the surface of the holder body opposite the cradles/opening, the holders can comprise mounting devices that allow for mounting of the holders to different surfaces including horizontal, angled, and vertical surfaces. This allows for mounting of the holders to the most convenient surface for use.

This application claims the benefit of U.S. Provisional Patent Application Ser. No. 63/057,438, filed on Jul. 28, 2020.

### BACKGROUND

### Field of the Invention

Described herein are devices relating syringes, and in particular, devices for safely and conveniently holding and organizing syringes during use.

### Description of the Related Art

Syringes are commonly used in many different medical procedures and more recently have become more commonly used in the beauty industry. For example, syringes can be used in the beauty industry to inject treatments, such as Botox, into the face or other body parts of a patient. These treatments can require several (or more) injections, and several syringes can be prefilled prior to the injection procedure.

Typically, the prefilled syringes can be placed on a tray or other surfaces in a medical office, and the practitioner can pick up the different desired syringes during the course of the treatment. The syringes held this way pose a risk of being injured by one or more of the syringe needles. This approach is also unorganized when using and replacing the syringes, and leaves the risk of used or unused syringes falling off the tray or surface to the ground.

Accordingly, there is a need to safely and securely hold these syringes in organized manner such that the danger of falling and dangers to the practitioner are reduced or eliminated. There is also a need for flexibility in mounting the syringes to different surfaces.

Different syringe holding devices have been developed such as the device produced by Aesthetic Practice Solutions, Inc., which comprises a solid block of material with rubber syringe cradles on different surfaces. These cradles, however, are arranged to hold the syringes in different orientations, such that the syringe needles are pointing in different directions. This results in a danger to the practitioner and/or assistants from being inadvertently injured by one of the syringes. This “block” approach also results in a device that can only be placed on a flat surface.

### SUMMARY OF THE INVENTION

The present invention is directed to simple, convenient, and cost-effective devices for holding syringes (“holder” or “holders”) in a manner to allow for easy access and use of the syringes by a practitioner. The holders according to the present invention can comprise a holder body with a plurality of cradles or openings for holding a syringe body, with the cradles/openings arranged to allow for fast and convenient mounting and removal of syringes to and from the holder. On the surface of the holder body opposite the cradles/opening, the holders can comprise mounting devices that allow for mounting of the holders to different surfaces including horizontal, angled, and vertical surfaces. This allows for mounting of the holders to the most convenient surface for use.

One embodiment of a syringe holding device according to the present invention comprises a body having a top surface. An array of linearly aligned syringe cradles are included on the top surface, wherein the cradles are arranged to hold syringes in the cradles in an orientation orthogonal to the linearly aligned cradles. Each of said cradles also comprises a syringe opening and at least two opposing flexible tabs. A mounting device is also included on the body and arranged to mount the device to a planar surface.

One embodiment of an office syringe holding system, comprising a planar surface within the office. A syringe holding device is included that is mounted to the planar surface. The syringe holding device comprises a body having a top surface. An array of syringe cradles is included on the top surface, wherein the body and cradles are arranged to hold syringes in the cradles in an orientation parallel to the planar surface. Each of the cradles comprise a syringe opening and at least two opposing flexible tabs. A mounting device is included on the body and arranged to mount the device to the planar surface.

These and other further features and advantages of the invention would be apparent to those skilled in the art from the following detailed description, taken together with the accompanying drawings, wherein like numerals designate corresponding parts in the figures, in which:

### BRIEF DESCRIPTION OF THE DRAWINGS

FIG. 1 is a top view of one embodiment of a syringe holder according to the present invention;

FIG. 2 is a side view of the syringe holder shown in FIG. 1;

FIG. 3 is a bottom view of the syringe holder shown in FIG. 1;

FIG. 4 is an end view of the syringe holder shown in FIG. 1;

FIG. 5 is a top view of another embodiment of a syringe holder according to the present invention;

FIG. 6 is a side view of the syringe holder shown in FIG. 5;

FIG. 7 is a bottom view of the syringe holder shown in FIG. 5;

FIG. 8 is an end view of the syringe holder shown in FIG. 5.

FIG. 9 is a top view of another embodiment of a syringe holder according to the present invention;

FIG. 10 is a side view of the syringe holder shown in FIG. 9;

FIG. 11 is an end view of the syringe holder shown in FIG. 9;

FIG. 12 is a side view of a portion of the syringe holder showing two cradles; and

FIG. 13 is side view of a portion of the syringe holder also showing two cradles.

### DETAILED DESCRIPTION

The present disclosure will now set forth detailed descriptions of various embodiments of syringe holding devices according to the present invention. As described in detail below, the holding devices provide a safe and secure way of holding the syringes that allow for safe and predictable access by the practitioner and/or assistants. The devises according to the present invention also comprise a means for securing the device in the desired location, including other horizontal surfaces. That is, the device can be mounted and held in place on angled or vertical surfaces, in the most convenient location for the practitioner.

In some embodiments according to the present invention, the device comprises a linear array of cradles, each of which is sized to hold a syringe. When the syringes are mounted in the device, they are arranged side by side, with the body of each syringe available for grasping. This allows for easy access to each of the syringes prior to use, and easy replacement of the syringe following use. By having all the syringes mounted side by side, the needles of the syringes are provided in a predictable orientation, which reduces the likelihood that a practitioner might inadvertently be injured by one of the syringes.

Throughout this description, the preferred embodiment and examples illustrated should be considered as exemplars, rather than as limitations on the present invention. As used herein, the term “invention,” “device,” “present invention,” or “present device” refers to any one of the embodiments of the invention described herein, and any equivalents. Furthermore, reference to various feature(s) of the “invention,” “device,” “present invention,” or “present device” throughout this document does not mean that all claimed embodiments or methods must include the referenced feature(s).

It is also understood that when an element or feature is referred to as being “on” or “adjacent” to another element or feature, it can be directly on or adjacent to the other element or feature or intervening elements or features may also be present. It is also understood that when an element is referred to as being “attached,” “connected” or “coupled” to another element, it can be directly attached, connected, or coupled to the other element or intervening elements may be present. In contrast, when an element is referred to as being “directly attached,” “directly connected” or “directly coupled” to another element, there are no intervening elements present.

Relative terms, such as “outer,” “above,” “lower,” “below,” “horizontal,” “vertical” and similar terms, may be used herein to describe a relationship of one feature to another. It is understood that these terms are intended to encompass different orientations in addition to the orientation depicted in the figures.

Although the terms first, second, etc. may be used herein to describe various elements or components, these elements or components should not be limited by these terms. These terms are only used to distinguish one element or component from another element or component. Thus, a first element or component discussed below could be termed a second element or component without departing from the teachings of the present invention.

The terminology used herein is for describing particular embodiments only and is not intended to be limiting of the invention. As used herein, the singular forms “a,” “an,” and “the” are intended to include the plural forms as well, unless the context clearly indicates otherwise. It will be further understood that the terms “comprises,” “comprising,” when used herein, specify the presence of stated features, integers, steps, operations, elements, and/or components, but do not preclude the presence or addition of one or more other features, integers, steps, operations, elements, components, and/or groups thereof.

Embodiments of the invention are described herein with reference to different views and illustrations that are schematic illustrations of idealized embodiments of the invention. As such, variations from the shapes of the illustrations as a result, for example, of manufacturing techniques and/or tolerances are expected. Embodiments of the invention should not be construed as limited to the particular shapes of the regions illustrated herein, but are to include deviations in shapes that result, for example, from manufacturing.

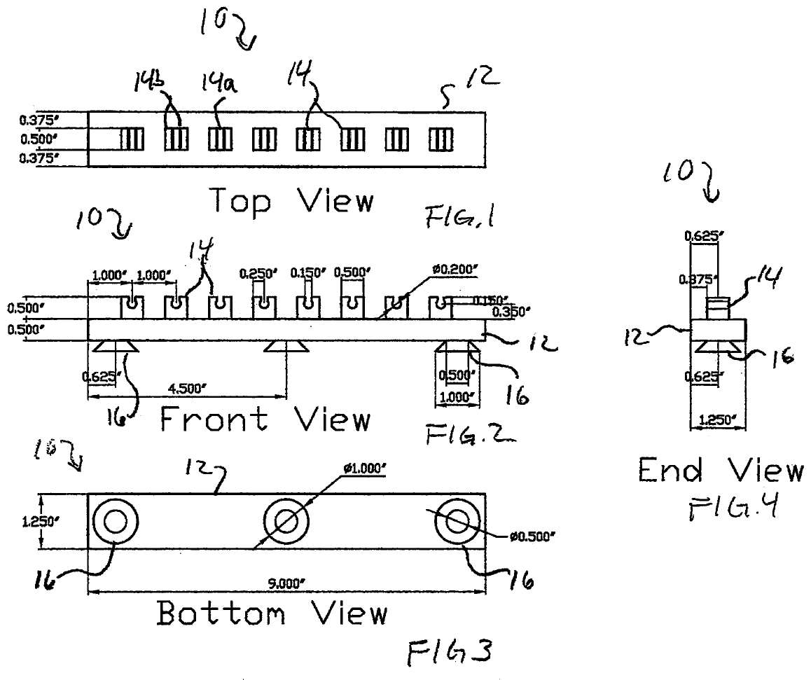

FIGS. 1-4 show one embodiment of a syringe holder 10 according to the present invention generally comprising an elongated body 12 made of rigid material, such as a metal or plastic. The body 12 can have many different shapes and sizes, with the body 12 in the embodiment shown having an elongated rectangular shape. The top surface of body 12 can be planar and can have a linear array of separate syringe cradles 14, each of which can be made of a material similar to the body 12. It is understood, however, that the cradles 14 can also comprise a different material, such as a rubber material.

Each of the cradles 14 is sized to hold the body of a syringe (not shown). In some embodiments, each cradle comprises an opening 14a, with tabs 14b formed on either side. A syringe can be mounted to each of the cradles 14 by holding the body of the syringe over the opening 14a and pushing the body into the opening 14a. This causes the tabs 14b to flex out and allow the body to pass into the base of the opening 14a. The tabs 14b then flex back to hold the syringe body in the opening 14a. The amount of flex in the tabs 14b can vary, with some embodiments having enough flex to allow for easy hand mounting of the syringe, but being rigid enough to firmly hold the syringe.

When a syringe is to be removed from a cradle 14, the syringe body can be grasped and pulled out of the particular cradle 14. This causes the tabs 14b to again flex out to open the cradle 14, allowing the syringe body to pass out of the cradle. Once the syringe is used, it can be returned to one of the cradles 14 in the same way as described above.

It is understood that the holders according to the present invention can have cradles with different sized openings to hold different sized syringes. The cradles need not be linearly aligned and in some embodiments may be offset. In still other embodiments, more than one cradle can be used to hold a single syringe body.

On the surface of the body 12 opposite the cradles 14, the holder 10 can comprise one or more mounting devices 16 that are designed and arranged to removably attach the holder 10 to a surface. Many different mounting devices 16 can be used in different embodiments of the present invention, with the embodiment shown having suction cups. The mounting devices 16 can provide a strong enough holding force so that the device can be mounted to and held to angled or vertical surface. The mounting devices 16 also help hold the holder 10 in place on horizontal surfaces. It is understood that many different mounting devices can be used in different embodiments, including but not limited to, adhesive, double sided tape, Velcro®, snaps, brackets, etc. It is also understood that other more permanent mounting devices can be used including bolts, screws, brackets, etc.

The present invention can be manufactured using many different methods, including injection molding of all or some of the parts. In other embodiments, all or some of the parts can be machined. In those embodiments where the different parts are manufactured using different methods, the parts can then be assembled together using adhesives or mechanical fasteners such as screws, bolts, rivets, nails, etc.

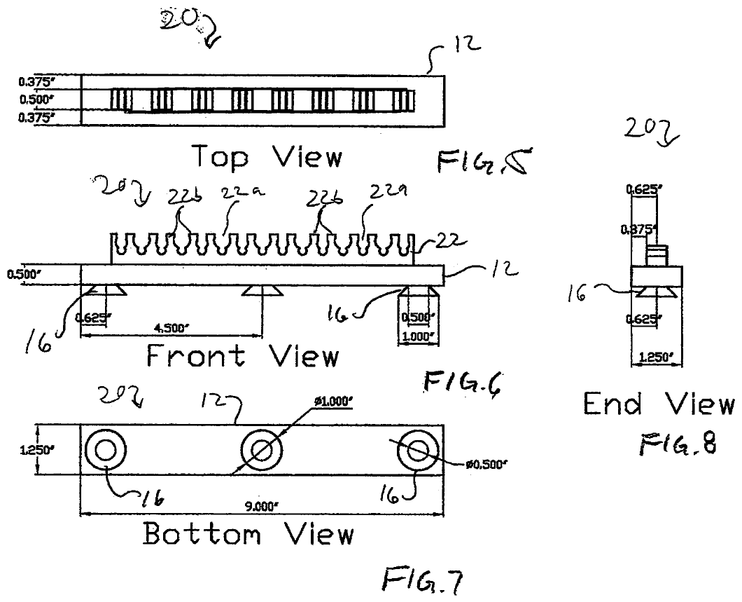

FIGS. 5-8 show another embodiment of a syringe holder 20 according to the present invention that is similar to the syringe holder 10 described above. For similar features the same reference number will be used to describe this embodiment with the understanding that the description above applies here. The holder 20 comprises a body 12 and mounting devices 16.

Instead of an array of cradles as described above, the holder 20 comprises a singular monolithic fixture 22 with a plurality of fixture openings 22a each of which is arranged to hold the body of a syringe. Each opening has a pair of tabs 22b that are similar to the tabs described above, and can flex when mounting or removing a syringe body to an opening. In this embodiment, the internal tabs can be shared between adjacent openings.

In the embodiment shown, the openings 22a have two different sizes for holding different sized syringes and in the fixture 22 the different sized openings alternate. It is understood that the fixtures according to the present invention can have even more different sized opening to hold two, three or more different sized syringes. It is understood that different sized syringes can have different lengths and/or different body widths.

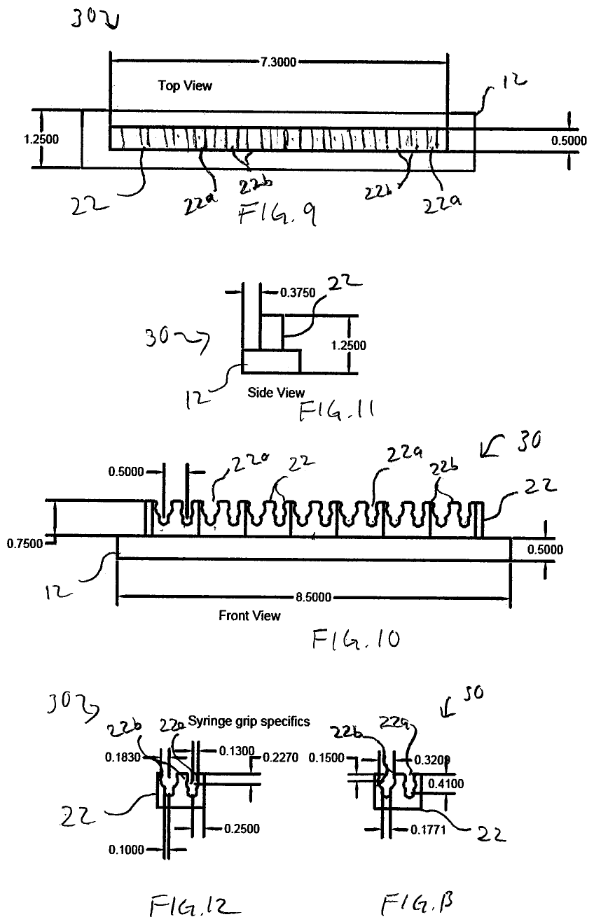

It is understood that different holders according to the present invention can be arranged in many different ways, with different dimensions and many different numbers of openings. FIGS. 9-13 show another embodiment of a syringe holder 30 according to the present invention that is similar to the syringe holder described above. Like holder 20, the holder 30 comprises a body 12, and a singular monolithic fixture 22 with a plurality of fixture openings 22a each of which is arranged to hold the body of a syringe. Each opening also has a pair of tabs 22b that are similar to the tabs described above, and can flex when mounting or removing a syringe body to an opening 22a. In this embodiment, the internal tabs can be shared between adjacent openings.

Like above, in this embodiment the openings 22a have two different sizes for holding different sized syringes and in the fixture 22 the different sized openings alternate. It is understood that the fixtures according to the present invention can have even more different sized opening to hold two, three or more different sized syringes.

In this embodiment however, the holder 30 has some slightly smaller dimensions and has one fewer syringe opening 22a, as shown in the drawings. For example, the length of the body 12 for holder 20 is 9.0 inches, while the length of the body 12 for holder 30 is 8.5 inches. Furthermore, there are fifteen syringe openings 22a for holder 20, and fourteen for holder 30. These are only some examples of the different dimensions that can be included in different holders according to the present invention.

It is understood that in different embodiments, the holders 20/30 (or alternatively holder 10 described above) can comprise more than one fixture 22/32, and each fixture 22/32 can comprise the same or different numbers of openings. By way of example only, different embodiments of the holder can comprise two fixtures, each of which can have the same or different numbers of opening with different sizes. It is also understood that more than one fixture can be used to hold a syringe. For example, holders according to the present invention can have two or more fixtures aligned with each other such the body of a syringe would be held in two or more fixture openings.

Although the present invention has been described in detail with reference to certain preferred configurations thereof, other versions are possible. Embodiments of the present invention can comprise any combination of compatible features shown in the various figures, and these embodiments should not be limited to those expressly illustrated and discussed. Therefore, the spirit and scope of the invention should not be limited to the versions described above.

The foregoing is intended to cover all modifications and alternative constructions falling within the spirit and scope of the invention, wherein no portion of the disclosure is intended, expressly or implicitly, to be dedicated to the public domain if not set forth in any claims.

We claim:

1. A syringe holding device, comprising:
a body comprising a top surface;
an array of linearly aligned syringe cradles on said top surface, wherein said cradles are arranged to hold syringes in said cradles in an orientation orthogonal to said linearly aligned cradles, wherein each of said cradles comprises a syringe opening and at least two opposing flexible tabs; and
a mounting device on said body and arranged to mount said device to a planar surface.

2. The holding device of claim 1, wherein said body is elongated.

3. The holding device of claim 1, wherein said body has an elongated rectangular shape.

4. The holding device of claim 3, wherein said top surface is planar with a first edge that is longer than a second edge, where and said cradles are aligned parallel to said first edge.

5. The holding device of claim 1, wherein said array of cradles comprises an array of separate cradles each of which hold a single syringe.

6. The holding device of claim 1, wherein said array of cradles comprises one or more monolithic fixtures, each of which has a plurality of syringe openings.

7. The holding device of claim 1, wherein said mounting device is on a surface of said body opposite said cradles.

8. The holding device of claim 1, wherein said mounting device is on a surface of said body other than a surface opposite said cradles.

9. The holding device of claim 1, wherein said mounting device comprises one or more suction cups.

10. An office syringe holding system, comprising:
a planar surface within said office;
a syringe holding device mounted to said planar surface, said syringe holding device comprising;
a body comprising a top surface;
an array of syringe cradles on said top surface, wherein said body and cradles are arranged to hold syringes in said cradles in an orientation parallel to said planar surface, wherein each of said cradles comprises and syringe opening and at least two opposing flexible tabs; and
a mounting device on said body and arranged to mount said device to said planar surface.

11. The holding system of claim 10, wherein said array of syringe cradles are linearly arranged.

12. The holding system of claim 11, wherein said linearly arranged cradles hold said syringes in an orientation orthogonal to said linear arrangement.

13. The holding system of claim 10, wherein said body is elongated and said top surface is planar with a first edge that is longer than a second edge, where and said cradles are aligned parallel to said first edge.

14. The holding system of claim 10, wherein said array of cradles comprises an array of separate cradles each of which hold a single syringe.

15. The holding system of claim 10, wherein said array of cradles comprises one or more monolithic fixtures, each of which has a plurality of syringe openings and is arranged to hold a plurality of syringes.

16. The holding system of claim 10, wherein said array of cradles is arranged to hold syringes having different sizes.

17. The holding system of claim 16, wherein said mounting device is on a surface of said body opposite said cradles.

18. The holding device of claim 10, wherein said mounting device comprises one or more suction cups.

19. A syringe holding device, comprising:
a body comprising a top surface and a bottom surface;
an array of linearly aligned syringe cradles on said top surface sized to hold a syringe, wherein each of said cradles comprises a syringe opening and at least two opposing flexible tabs, and wherein different ones of said cradles are arranged to hold syringes of different sizes; and
a suction cup mounting device on said bottom surface.

20. The holding device of claim 19, wherein said cradles are arranged to hold syringes in said cradles in an orientation orthogonal to said linearly aligned cradles.
